(19) United States
(12) Patent Application Publication
(10) Pub. No.: US 2025/0108142 A1
(43) Pub. Date: Apr. 3, 2025

(54) METHODS FOR TREATING LEAKAGE FROM A GASTROINTESTINAL SITE

(71) Applicant: Ethicon, Inc., Raritan, NJ (US)

(72) Inventors: Degang Zhong, Branchburg, NJ (US); Gerard Llanos, Stewartsville, NJ (US); Jianguo Jack Zhou, Allentoen, PA (US)

(21) Appl. No.: 18/374,019

(22) Filed: Sep. 28, 2023

(51) Int. Cl. A61L 24/04; A61K 31/192; A61K 31/496

(52) U.S. Cl. CPC A61L 24/043; A61K 31/192; A61K 31/496; A61L 2300/204; A61L 2300/21; A61L 2300/406; A61L 2300/41; A61L 2400/06

(57) ABSTRACT

Disclosed herein are flowable biocompatible sealant compositions in the form of a biodegradable cross-linked gel, comprised of an insoluble agent for preventing infection, and an agent for preventing ischemia, wherein the agent for preventing ischemia is embedded in polymeric microparticles. The composition is characterized in that it releases at least part of said agents in a sustained manner. Uses of the compositions in methods for treating a leakage from a gastrointestinal site are further disclosed.

### FIELD OF THE INVENTION

The invention relates, inter alia, to compositions and methods for preventing and/or treating leakage from a gastrointestinal site.

### BACKGROUND OF THE INVENTION

The gastrointestinal (GI) tract extends from the esophagus to the anus and senses many functions, including nutrition, hydration, and disease prevention. A GI leak is a critical post-operative complication of procedures related to surgical anastomosis of the GI tract and is substantial risk of the patient leaking at the site of the anastomosis even if the surgeon follows best practices. Anastomotic leakage has been the bane of intestinal surgery for over a century.

High leak rates complicate about 3.5 million colorectal surgeries/year world-wide. Patients with anastomotic leaks have increased lengths of stay, mortality rates, readmission rates, and need for reoperation with resulting negative impact on quality of life. The leak rate for GI anastomosis varies from 1 to 20% depending on the location, procedure and other risk factors and has not changed significantly for 40 years. In addition, there is no clinically representative preclinical model of anastomotic leaks. Although various surgical techniques and interventions have been developed to mitigate leaks, no effective solution is currently available.

U.S. patent publication No. discloses a composition comprising a polymer and at least one active agent, wherein the composition is formulated for topical application and shows thermally reversible behavior or inverse thermally reversible behavior. The active agent of the composition is an antimicrobial, an anti-inflammatory agent, anesthetic or mixtures thereof.

U.S. Pat. No. 8,894,699 discloses methods and apparatus for anastomosis of a lumen according to various aspects of the present invention operate in conjunction with an impermeable stent. The impermeable stent may comprise a scaffold and, if needed, a sealant, such as a membrane and/or adhesive. In one embodiment, the scaffold, membrane and/or adhesive comprise biocompatible materials suitable for bio-absorption and/or degradation.

### SUMMARY OF THE INVENTION

The invention relates, inter alia, to a method for preventing and/or treating leakage from a gastrointestinal (GI) site in a subject in need thereof.

An object of the present invention is to provide a delivery matrix sealant that can deliver a combination of two pharmacological agents into the GI site in a sustained manner for at least two weeks, to address both infection and ischemia. The delivery matrix may include a biologic (e.g., fibrin sealant) or synthetic hydrogel (e.g., PEG-based sealant), for locally applying to the anastomosis site.

The reasons for GI leaks are multifactorial. Infection and ischemia have been identified as two leading causes. An underlying concept of the present disclosure is to use a fibrin or synthetic based sealant to deliver pharmacologically or biologically active agents that can address both issues. To achieve this, in exemplary embodiments, ciprofloxacin was chosen as the antibiotic agent and ibuprofen was chosen as a nonsteroidal anti-inflammatory (NSAID) and anti-coagulant drug. Ciprofloxacin is practically insoluble in water and ibuprofen has minimum solubility in water. They can be mixed in suspension in fibrinogen component of a fibrin sealant such as EVICEL®. A fibrin sealant comprising fibrinogen: thrombin (e.g., 5:1 by volume), may be delivered onto anastomosis site by 5:1 device to form fibrin sealant barrier. The fibrin sealant may stay on site for a couple of weeks. Meanwhile the drugs (e.g., ciprofloxacin and ibuprofen) are released in a sustained manner by diffusion in fibrin sealant onto the surgical site. Antibiotics can prevent infection on site; and ibuprofen can reduce ischemia and also act as a nonsteroidal anti-inflammatory drug on a surgical site. Since ibuprofen is slightly soluble in water (about 21 mg/l), it can affect sealant or fibrin clot formation. Therefore, ibuprofen may also be embedded into a polymer (e.g., poly (lactic-co-glycolic acid); PLGA) to produce small particles to reduce the interruption of clot formation and to allow a sustained release manner. Alternatively, a synthetic sealant (made from e.g., PEG Succinimidyl Glutarate (PEG-SG) and PEG-NH2) may be used instead of fibrin sealant.

According to an aspect of the present disclosure, there is provided a flowable biocompatible sealant composition in the form of a biodegradable cross-linked gel, the composition comprising an insoluble agent for preventing infection, and an agent for preventing ischemia; wherein:
(a) the agent for preventing ischemia is embedded in polymeric microparticles.
(b) the composition is characterized by adhesiveness within the range of 0.1 kPa to 100 kPa as measured by a tack test based on ASTM F2258-05; and wherein
(c) the composition is characterized in that it releases at least part of the agents in a sustained manner.

According to another aspect of the present disclosure, there is provided a method for preventing and/or treating leakage from an anastomosis site in a subject in need thereof, the method comprising the steps of:
(a) providing a flowable biocompatible sealant composition capable of forming a biodegradable gel, e.g., cross-linked gel), the composition comprising a therapeutically effective amounts of: an agent for preventing infection, and an agent for preventing ischemia;
(b) applying the composition of step (a) to the anastomosis site; and
(c) allowing the composition to form a gel, and to locally release at least part of the two active agents through diffusion and/or upon biodegradation of the gel.

In some embodiments of any aspect, the gel comprises fibrin.

In some embodiments of any aspect, the gel comprises Polyethylene glycol (PEG) or a derivative thereof, such as functionalized PEG.

In some embodiments of any aspect, the functionalized PEG is in a multi-arm form.

In some embodiments of any aspect, the insoluble agent for preventing infection comprises ciprofloxacin.

In some embodiments of any aspect, the agent for preventing ischemia comprises nonsteroidal anti-inflammatory agent.

In some embodiments of any aspect, the anti-inflammatory agent comprises ibuprofen.

In some embodiments of any aspect, the agent for preventing ischemia is homogenously embedded in polymeric microparticles.

In some embodiments of any aspect, the polymeric microparticles are made of polylactic-co-glycolic acid (PLGA) or gelatin.

In some embodiments of any aspect, the composition comprises at least one portion exhibiting a release profile of the agent for preventing ischemia that is representative of first-order release kinetics under in vitro conditions.

In some embodiments of any aspect, the flowable biocompatible sealant composition is provided in a kit or a delivery system.

In some embodiments of any aspect, the flowable biocompatible sealant composition is provided in a two-component delivery system: component A comprising: one member selected from electrophilic functionalized PEG, the active agent(s), and fibrinogen and Component B comprising one member selected from: nucleophilic functionalized PEG, the active agent(s) and thrombin.

In some embodiments of any aspect, the electrophilic functionalized PEG has attached thereto one or more succinimidyl glutarate groups and the nucleophilic functionalized PEG has attached thereto one or more amine groups.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 presents a photographic image of a 5:1 device of a sealant applicator. In exemplary procedures fibrinogen is loaded into the 5 ml syringe and thrombin is loaded into the 1 ml syringe. The device is operated by pushing the plunger-connector to apply the sealant to a target site.

FIG. 2 presents a microscopic image of polylactic-co-glycolic acid (PLGA)-Ibuprofen microparticles as prepared in exemplary procedures.

FIGS. 3A-3B present graphs showing individual daily amount (FIG. 3A) and total (accumulative) amount (FIG. 3B) of ciprofloxacin released from fibrin clots into the solution (in an in vitro setting) as shown in Table 1.

FIGS. 4A-4B present graphs showing individual daily amount (FIG. 4A) and total (accumulative) amount (FIG. 4B) of PLGA-Ibuprofen from fibrin clots released into the solution (in an in vitro setting) as shown in Table 2B.

FIGS. 5A-5B present graphs showing individual daily amount (FIG. 5A) and total (accumulative) amount (FIG. 5B) of ciprofloxacin released from a gel made of a polymer comprising polyethylene glycol (PEG) into the solution (in an in vitro setting) as shown in Table 3.

FIGS. 6A-6B present graphs showing individual daily amount (FIG. 6A) and total (accumulative) amount (FIG. 6B) of PLGA-Ibuprofen from PEG gels released into the solution (in an in vitro setting) as shown in Table 4B.

### DESCRIPTION OF EMBODIMENTS OF THE INVENTION

The invention relates, inter alia, to compositions and methods for preventing and/or treating leakage from a gastrointestinal (GI) site.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

The present inventors have developed specific compositions for treating GI and anastomotic leak. The compositions may be in the form of a gel made from biocompatible polymers which have a sufficient adhesiveness to stay at the desired site of a GI tissue while locally and effectively releasing a combination of two different active agents in a time controlled and/or sustained release manner.

One key advantage of this controlled release is that it inherently improves efficacy of the treatment and decreases potential side effects, when compared to other routes of administration such as oral, rectal, topical, or systemic.

Specifically, the present inventors have developed a flowable biocompatible sealant composition in the form of a biodegradable cross-linked gel made of a polymeric matrix, the composition comprising an insoluble agent for preventing infection e.g., antibiotic, and an agent for preventing ischemia, e.g., nonsteroidal anti-inflammatory agent; wherein the agent for preventing ischemia (which is at least slightly soluble, e.g., Ibuprofen having a reported solubility of 21 mg/L) is embedded in insoluble polymeric (e.g., PLGA) microparticles so that this agent is microencapsulated in the non-solubilized matrix, allowing its sustained release. Thus, the composition is characterized in that it releases at least part of the agents in a sustained manner.

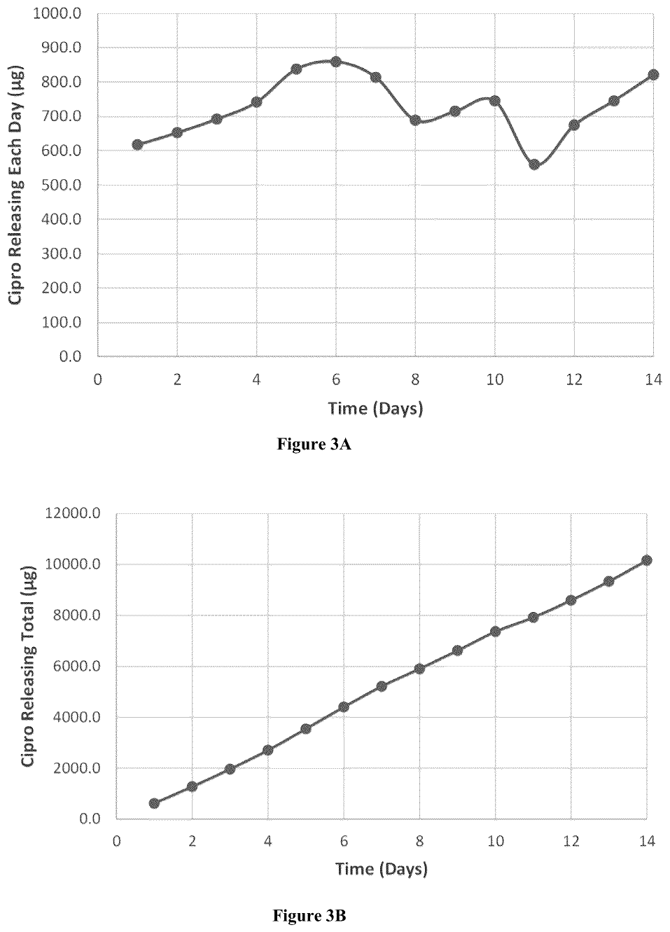

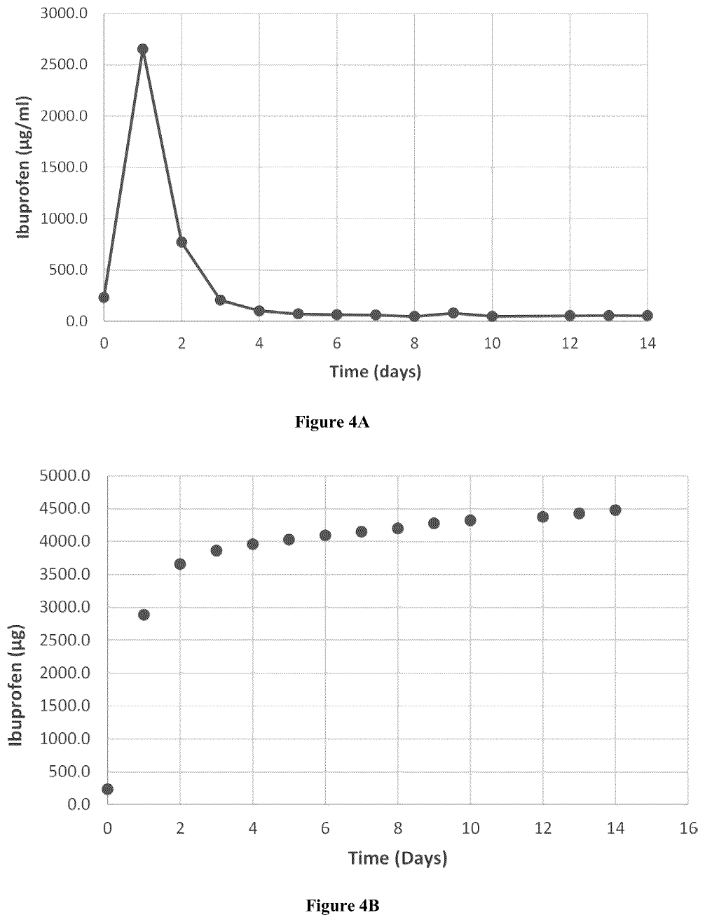

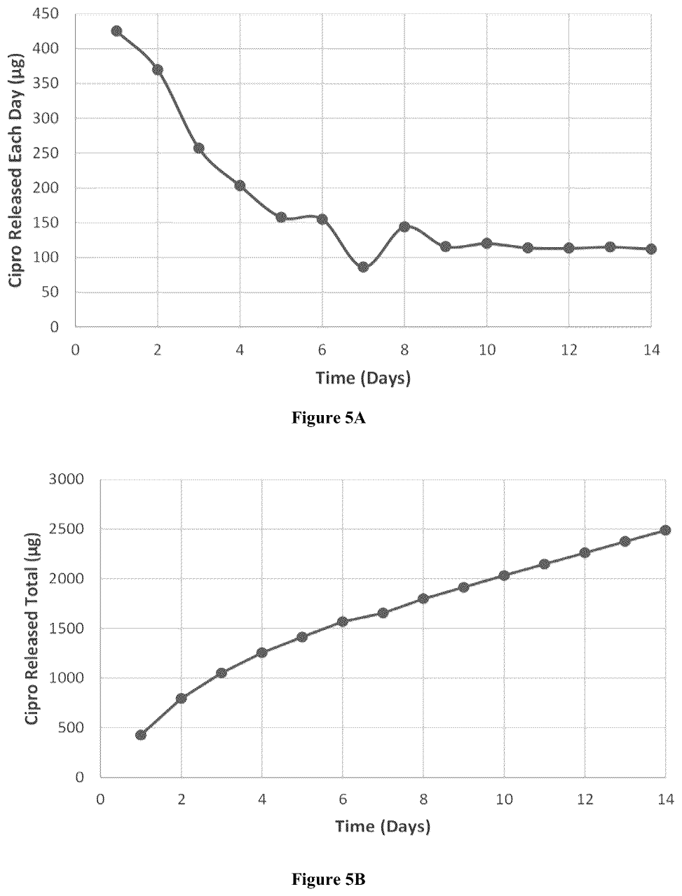

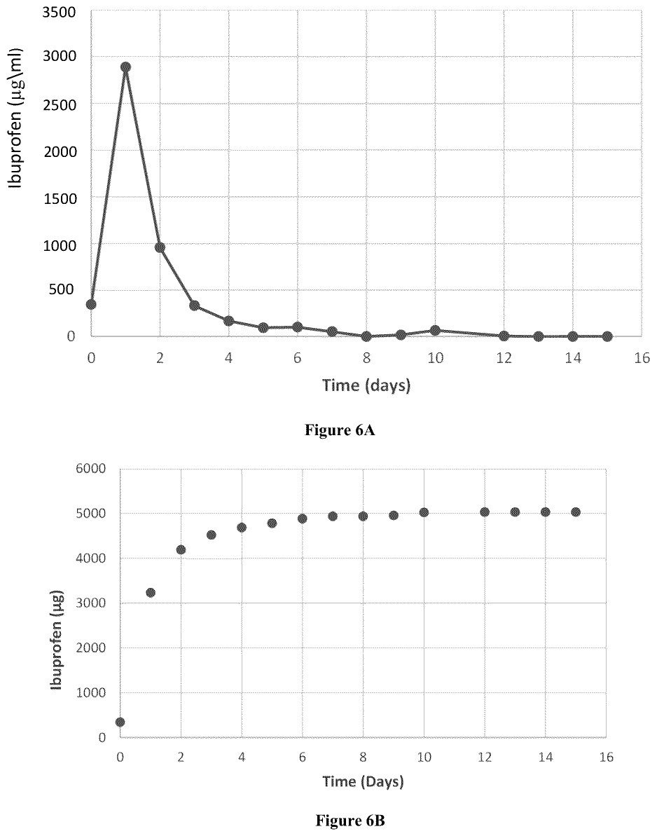

As is illustrated in the Examples section which follows, ciprofloxacin was consistently at an approximate zero-order release profile both from a fibrin clot (see FIGS. 3A and 3B) and from a polyethylene glycol (PEG) gel (see FIGS. 5A and 5B). Zero-order release kinetics refers to the process of constant drug release from a drug delivery system resulting in drug levels that would remain constant throughout the delivery period. The release profile of PLGA-Ibuprofen was consistently at an approximate first-order release profile both from a fibrin clot (see FIGS. 4A and 4B) and from a PEG gel (see FIGS. 6A and 6B).

Consequently, specific embodiments of the present invention propose novel compositions and their use in anastomotic leak therapy in a subject in need thereof.

As used herein “anastomotic leak” refers to a defect of the intestinal wall at or near the anastomotic site (including suture and staple lines of neorectal reservoirs), leading to a communication between the intra- and extraluminal compartments. A pelvic abscess close to anastomosis is also considered an anastomotic leak.

According to an aspect of some embodiments of the present disclosure, there is provided a flowable biocompatible sealant composition in the form of a biodegradable cross-linked gel, the composition comprising an insoluble agent for preventing infection, and an agent for preventing ischemia; wherein:
(a) the agent for preventing ischemia is embedded in polymeric microparticles.
(b) the composition is characterized by adhesiveness within the range of 0.1 kPa to 100 kPa as measured by a tack test based on ASTM F2258-05; and wherein:
(c) the composition is characterized in that it releases at least part of the agents (i.e. one of them or both) in a sustained manner.

In another aspect of the present disclosure the invention relates to a method for preventing and/or treating leakage from a gastrointestinal site in a subject in need thereof, the method comprising the steps of:
(a) providing a flowable biocompatible sealant composition capable of forming a biodegradable gel, the composition comprising one or more active agents;
(b) applying the composition of step (a) to the gastrointestinal site; and
(c) allowing the composition to form a gel, and to locally release at least part of the one or more active agents through diffusion and/or upon biodegradation of the gel.

Typically, but not exclusively, one or more active (also referred to as: “therapeutic”) agents are loaded into the polymer matrix in an amount of 1% to 25% of the total weight of the polymer matrix material.

In a further embodiment of the invention, the one or more active agents are selected from an antibiotic, anti-ischemic, anti-infective, anti-coagulant agent, or any combination thereof. In some embodiments, the composition comprising an agent for preventing ischemia comprises an anti-inflammatory agent. In some embodiments, the term “anti-inflammatory agent” refers to a non-steroidal anti-inflammatory drug.

Non limiting examples of the one or more active agents are selected from non-steroidal anti-inflammatories such as, without being limited thereto, acetyl salicylate, ibuprofen, naproxen or diclofenac and broad-spectrum antibiotics, such as without being limited thereto, quinolones (e.g., ciprofloxacin), aminoglycosides, ampicillin, amoxicillin/clavulanic acid, carbapenems piperacillin/tazobactam, tetracyclines, chloramphenicol, ticarcillin, and trimethoprim/sulfamethoxazole.

More specific non-limiting examples of one or more active agents are: non-steroidal anti-inflammatories such as acetyl salicylate, ibuprofen, naproxen or diclofenac.

Further embodiments of active agents are described hereinthroughout.

The term “cross-linked” herein refers to a composition containing intermolecular cross-links and/or intramolecular cross-links, e.g., arising from the formation of covalent bonds.

The term “anti-coagulant agent” as used herein may include any agent which allows to avoid or slow down the formation of a blood clot.

In the scope of the present disclosure, the term “flowable” is understood to mean liquid materials, gelled composition or viscous materials, and even highly viscous materials, that flow only upon the application of pressure. The term “viscous solution” refers to a solution that has an increased resistance to flow, yet is capable of flowing to some extent.

In the context of the present invention, the term “sealant”, also referred to as “biological glue” is to be understood as an adhesive, glue, or hemostat, e.g., such as one originates or being derived from the disclosed composition or components thereof, having ingredients that upon contact with each other or with, or in proximity to, a tissue and/or blood, reacts to subsequently form a clot, and may further act as a tissue adhesive, and thereby prevents, reduces, or stops bleeding, joins structures and/or seals physiological leaks, e.g., of gastrointestinal (GI) content, cerebrospinal fluids (CSF), lymph, bile, air leak from lungs etc. In some embodiments, the sealant formulation also comprises one or more therapeutics disclosed herein, such that upon natural degradation of the composition, the therapeutic is released.

The term “preventing leaks” as used herein includes administering to a subject prone to such leakage an effective amount of the flowable biocompatible sealant composition comprising one or more active agents. The term “treating leaks” as used herein includes administering to a subject afflicted with such leaks an effective amount of the flowable biocompatible sealant composition comprising one or more active agents.

The term “adhesiveness” refers to pressure-sensitive adhesiveness or tackiness that has tackiness and adhesion to a surface of an organ to the extent that it does not easily slip off the affected area. Methods for measuring adhesiveness are known in the art, e.g., ASTM F2258-05, which is a Standard Test Method for Strength Properties of Tissue Adhesives in Tension. ASTM F2258-05 test method is intended to provide a means for comparison of the adhesive strengths of tissue adhesives intended for use as surgical adhesives or sealants, or both, on soft tissue.

In some embodiments, the composition is characterized by adhesiveness within the range of 0.1 kPa to 100 kPa as measured by a tack test, including any value and range therebetween. In some embodiments, the composition is characterized by adhesiveness within the range of 0.1 kPa to 1 kPa as measured by a tack test. In some embodiments, the composition is characterized by adhesiveness within the range of 1 kPa to 10 kPa as measured by a tack test. In some embodiments, the composition is characterized by adhesiveness within the range of 0.1 kPa to 10 kPa as measured by a tack test. In some embodiments, the composition is characterized by adhesiveness within the range of 10 kPa to 100 kPa as measured by a tack test.

By “agent” or “active agent” it is meant an entity, a substance or a chemical capable of producing an effect. The agent may be a pharmaceutical drug, a substance, such as a contrasting agent to be used for diagnostic or, a nutritional substance.

The term “insoluble agent” (also referred to as “loosely soluble agent”) as used herein is as defined in the U.S. Pharmacopeia, Description and Relative Solubility of USP and NF Articles. That is, this term relates to either “sparingly soluble”, “slightly soluble”, “very slightly soluble”, or “practically insoluble, or insoluble” as defined therein. Accordingly, in terms of parts of solvent required for 1 part of solute, the term “insoluble agent” relates to from 30 to 100, from 100 to 1,000, from 1,000 to 10,000, or greater than or equal to 10,000. Herein, an expression like “100 to 1,000” means that 100 parts by volume of a liquid shall be diluted with, or 100 parts by weight a solid shall be dissolved in a sufficient quantity of the diluent or solvent (e.g., water) to make the volume of the finished solution 1,000 parts by volume.

In some embodiments, the term “insoluble agent” relates to a solubility in an aqueous solution of less than <1 mg/mL, less than 100 μg/mL, less than 10 μg/mL, less than 5 μg/mL or less than 1.5 of μg/mL.

In some embodiments, the insoluble agent for preventing infection comprises an anti-infective agent. “Anti-infective agent” as used herein covers agents that are, e.g., capable of killing, inhibiting or otherwise slowing the growth of infectious agent such as viruses, fungi, and bacteria. Anti-infective agents may include broad-spectrum antibiotics. The term “antibiotic” or “antibiotic agent” as used herein includes a drug intended for the treatment of infectious disease, such as any drug intended for human use containing any quantity of any chemical substance which may be produced by a microorganism, and which has the capacity to inhibit or destroy microorganisms in a dilute solution.

Exemplary antibiotic agents include, without limitation, quinolones (e.g., ciprofloxacin), aminoglycosides, ampicillin, amoxicillin/clavulanic acid, carbapenems piperacillin/tazobactam, tetracyclines, chloramphenicol, ticarcillin, and trimethoprim/sulfamethoxazole.

In exemplary embodiments, the insoluble agent for preventing infection comprises ciprofloxacin. Ciprofloxacin is a broad-spectrum second generation 4-fluoroquinolone antibiotic that targets the bacterial enzyme DNA gyrase and is effective against Gram-positive and Gram-negative bacteria and mycoplasmas.

As used herein the term “ischemia” refers to the damage caused first by restriction of the blood supply to a tissue followed by a sudden resupply of blood.

The term “agent for preventing ischemia”, or “anti-ischemic agent”, is used to refer to a drug or therapeutic agent that reduces tissue damage due to ischemia and/or low or no reperfusion.

In some embodiments of the method, the composition comprises a therapeutically effective amount of an agent for preventing infection, and an agent for preventing ischemia.

In some embodiments, the release of an active agent from the disclosed composition is affected in-vitro or ex-vivo. In some embodiments, the release of an active agent from the disclosed composition is affected in-vivo.

As used herein, the term “subject” shall mean any animal including, without limitation, a human, a mouse, a rat, a rabbit, a non-human primate, or any other mammal. In some embodiments, the subject is human, e.g., a human patient. The subject may be male or female. In one embodiment, the subject in need is a patient.

The term “biocompatible” as used herein includes ability to perform its function in the body with an acceptable biological response. The term “biodegradable” as used herein includes a material susceptible or able to be degraded or disintegrate in vivo.

The term “composition” as used herein includes a substance or preparation formed by combination or mixture of various ingredients. In some embodiments, the composition comprises a homogenous suspension, formulation or gel. As used herein, by “homogeneous” it is meant to refer to a uniform suspension throughout, e.g., having a density and/or viscosity that vary within a range of ±25%.

The term “release” as used herein includes to free from something that binds, fastens, or holds back, or are solubilized.

Herein, the term “sustained-release” relates to a composition that release the active agent over a period of time greater than 24 hours (h) or longer, e.g., about 1 day, about 2 days, about 3 days, about 4 days, about 5 days, about 6 days, about 7 days, about 15 days, about 30 days, about 45 days, about 60 days, about 75 days, or about 90 days after administration, including any value and range therebetween.

The term “diffusion” as used herein includes the motion of an agent as dictated by local concentration gradient conditions.

The term “solubilized” as used herein includes an agent being dissolved in an aqueous medium.

The term “gel” as used herein relates to a material which has properties of the solid state and under certain conditions exhibits properties of the liquid state, for example, a hydrogel.

In some embodiments, the gel comprises a biological sealant. A biological sealant composition relates, e.g., to a composition comprising fibrinogen, thrombin or fibrin.

As used herein, “thrombin” denotes an activated enzyme which results from the proteolytic cleavage of prothrombin (factor II). Thrombin may be produced by a variety of methods of production known in the art, and includes, but is not limited to, recombinant thrombin and plasma derived thrombin.

Human thrombin is a 295 amino acid protein composed of two polypeptide chains joined by a disulfide bond. Both human and non-human (e.g., bovine) thrombin may be used within the scope of the present disclosure.

The term “fibrinogen” may include any type of fibrinogen such as, without limitation, fibrinogen in a cryoprecipitate. Fibrinogen, therefore, refers to monomeric and dimeric fibrinogen molecules having the monomer structure (AαBβy), hybrid molecules, and variants thereof, whether from plasma, modified, or recombinant. The term “fibrinogen” refers generally to fibrinogen from humans, but may include fibrinogen of any species, especially mammalian species.

Any preparation for therapeutic use must be sterile. Especially when handling blood products, the sterility issue is crucial, and specifically the issue of viral inactivation. In general, viral inactivation may be carried out by any number of methods, including solvent detergent, heat inactivation, irradiation, and nanofiltration, and the standard for viral inactivation requires using two different methods. Additionally, the U.S. Food and Drug Administration (FDA) standard for sterility requires filtration.

The term “liquid fibrin sealant formulation” as used herein below refers to at least two separated liquid components required for the formation of fibrin sealant, e.g., a fibrinogen-comprising component and a thrombin-comprising component. When the at least two separated components are administered e.g., injected into/onto the gastrointestinal site, the liquid components come into contact and form a gel (or a fibrin clot).

The gastrointestinal site may comprise a gastrointestinal anastomotic surgery site from the esophagus through to the anal verge: e.g., esophageal anastomosis and/or lower anterior resection of the rectum.

The fibrinogen-comprising component can be comprised of a biologically active component (BAC) which is a solution of proteins derived from blood plasma. BAC can be derived from cryoprecipitate, such as concentrated cryoprecipitate. The term “cryoprecipitate” refers to a blood component which is obtained from frozen plasma prepared from whole blood. A cryoprecipitate can be obtained when frozen plasma is thawed in the cold, typically at a temperature of 0-4° C., resulting in the formation of precipitated supernatant that contains fibrinogen and factor XIII. The precipitate can be collected, for example by centrifugation. Typically, BAC comprises Factor VIII, fibronectin, von Willebrand factor (vWF), vitronectin, etc. for example as described in U.S. Pat. No. 6,121,232 and corresponding published PCT application WO 98/33533, each incorporated herein by reference.

In some embodiments of any aspect, BAC refers to Biologically active component 2 (BAC2), i.e. a biologically active component that lacks tranexamic acid. BAC2 is a concentrated viral-inactivated cryoprecipitate of human plasma (the cryoprecipitate is typically prepared as described in EP 534,178) which comprises mainly of fibrinogen (e.g., approx. 85%) and is plasminogen-depleted (the removal of plasminogen is typically carried out as described in EP 1,390,485) and without anti-fibrinolytic agents added. In view of removal of plasmin/plasminogen from the cryoprecipitate, there is no need to add anti-fibrinolytic agents, such as tranexamic acid, aprotinin or the like.

Optionally, the solution is buffered to a physiological compatible pH value. The buffer may be comprised of glycine, sodium citrate, sodium chloride, calcium chloride and water for injection as a vehicle. Glycine may be present in the composition in the amount of from about 6 to about 10 mg/ml, the sodium citrate may be in the range of from about 1 to about 5 mg/ml, sodium chloride may be in the range of from about 5 to about 9 mg/ml and calcium chloride may be in the concentration of about 0.1-0.2 mg/ml.

It is also possible that the fibrin sealant formulation comprises components which encourage the formation of the clot, such as Ca²⁺, Factor VIII, fibronectin, vitronectin, von Willebrand factor (vWF) which can be provided as a separate component or formulated with the liquid components.

The liquid sealant formulation may be applied onto the tissue using a dispenser that ejects the sealant directly onto the tissue or other substrate or working surface with or without air spray e.g., by dripping. Examples of tissue sealant dispensers are shown in U.S. Pat. Nos. 4,631,055, 4,846,405, 5,116,315, 5,582,596, 5,665,067, 5,989,215, 6,461,361 and 6,585,696, 6,620,125 and 6,802,822, and PCT Publication No. WO 96/39212, WO 2007/059801, and WO 2010/095128, all of which are incorporated herein by reference.

During surgical operations, the separated liquid components e.g., two liquid components, are applied, for example, by two syringes which are emptied simultaneously or one after the other resulting in mixing of the two components and formation of fibrin.

Thrombin in the thrombin-comprising component of the fibrin sealant may have an activity of from about 2 to about 4,000 IU/ml. The clotting activity of thrombin can be measured directly, for example, according to the European Pharmacopoeia Assay (0903/1997) procedure and/or indirectly, such as by measuring migration length on a slanted surface (e.g., the “drop test” as described in published US patent application 2010/0203033) or by any other method known in the art.

In a further embodiment of the disclosure, the sealant is comprised of a two-component sealant, with the one or more active agents being present in one or both components.

Fibrin sealant components, whether as part of the two-component liquid fibrin sealant or the solid fibrin sealant blend, derived from blood or blood fractions are typically subjected to at least two discrete virus inactivation/removal steps. Virus inactivation and removal processes can be carried out e.g., by the following methods: nanofiltration; solvent/detergent; low pH treatment, UV irradiation; sodium thiocyanate treatment and/or by any other method known in the art.

Fibrinogen- and thrombin-containing components e.g., EVICEL® are available.

During application of the liquid fibrin sealant formulation onto the tissue, the fibrinogen-comprising component and the thrombin comprising component may be applied in any desired range of ratios. For example, when the concentration of the fibrinogen component is 40-85 mg/ml and the thrombin concentration is about 800-1200 IU/ml, the two components may be mixed in a ratio of e.g., 1:1, 1:2, 1:3, 1:4, 1:5, or 1:6, respectively, by volume, including any value and range therebetween. In one embodiment of the invention, the components of the liquid fibrin sealant are applied in a ratio of about 1:5, respectively.

In one embodiment, the liquid components are applied simultaneously. Yet in another further embodiment, the liquid components are applied one after the other. The fibrin sealant may be applied extraluminally and/or endoluminally. The sealant composition may be applied in a volume range of from about 0.01 to about 1 ml per cm² such as in a volume of 0.01, 0.05, 0.5 or 1 ml per cm², including any value and range therebetween.

In one example, for a 10 mm incision, the fibrin formulation is applied by spraying a total volume of 2 ml 1000 IU/ml thrombin and 70 mg/ml fibrinogen.

In one embodiment of the invention, less than 50% of the gel is biodegraded over a period within the range of 1 to 90 days after the application. In one embodiment of the invention, less than 50% of the gel is biodegraded over a period within the range of 1 to 10 days after the application.

The term “locally” as used herein includes the region in which the gel was formed in or administered to in the body.

The term “anastomosis” as used herein includes the surgical connection between the ends of the lumen in a surgery site. This term typically refers to a surgical procedure which is used to reconnect two or more sections of an organ or tissue. The procedure can be used following sectioning of the bowel during bowel surgery. The procedure can also be used following the excision of a diseased tissue (such as inflamed, cancerous or otherwise pathological tissue e.g., ulcerative disease) and/or defects such as intestinal perforation.

In some embodiments, the sealant composition is made from synthetic or semi-synthetic composition.

The term “synthetic composition” as used herein relates to a not-naturally occurring composition.

In some embodiments, the gel comprises polyethylene glycol (PEG) or a derivative thereof (e.g., functionalized PEG). In some embodiments, the gel comprises PEG or a derivative thereof and fibrinogen. In some embodiments, the gel comprises PEG or a derivative thereof and fibrinogen and one or more active agents described herein.

The synthetic sealant composition may be two-part, two-component, or co-reactant compositions comprising a electrophilic co-reactant such as, without limitation, multi-N-hydroxysuccinimide (NHS) functionalized polyalkylene glycol co-reactant (e.g., polyethylene glycol-NHS) and a nucleophilic co-reactant such, without limitation, as multi-arm amine functionalized polyalkylene glycol co-reactant (e.g., polyethylene glycol amine) or a NHS derivatized polyoxazolines co-reactant and amine derivatized polyoxazolines co-reactant or combinations thereof.

The two components may be mixed in a ratio of e.g., 6:1, 5:1, 4:1, 3:1, 2:1, 1:1, 1:2, 1:3, 1:4, 1:5, or 1:6, including any value and range therebetween.

Other non-limiting examples include two-part systems that combine an oxidized polysaccharide-aldehyde (e.g., dextran-aldehyde) and a polyamine such as a multi-arm amino polyalkylene glycol or polyethylene imine. The synthetic sealant may also be a single reactive composition that forms a sealant barrier upon receiving an external stimulus. Non-limiting examples includes isocyanate end-capped, multi-arm polyalkylene-glycols which react with aqueous media to form a hydrogel. Another non-limiting example is acryloyl end-capped multi-arm polyethylene glycol which upon exposure to light of a particular wavelength (e.g., UV) will form a suitable sealant gel.

Other non-limiting examples of synthetic composition comprise polyoxazoline. The term “polyoxazoline” as used herein refers to a poly(N-acylalkylenimine) or a poly(aroylalkylenimine) and is further referred to as “POX”. The term “polyoxazoline” as used herein also encompasses POX copolymers. In some embodiments, the POX polymer comprises at least 30 oxazoline units in case the electrophilic cross-linking agent is an isocyanate.

Semi-synthetic sealant composition may be comprised from a two-part co-reactant composition consisting e.g., of a multi-N-hydroxysuccinimide (NHS) functionalized polyalkylene glycol co-reactant (e.g., polyethylene glycol-NHS) and a protein such as serum albumin. In some embodiments, the cross-linking is between PEG-NHS ester and PEG-amine or trilysine.

In exemplary embodiments, the polymer comprises PEG Succinimidyl Glutarate (PEG-SG), e.g., at least two different multi-arm PEG-SG components.

The active agents may be present in the sealant composition at an amount higher than the amount needed for obtaining a solubilized active agent in a given volume of the sealant composition forming a gel. Typically, most of the active agent is present in the non-solubilized form. Advantageously, the agent is in the form of homogenous suspension and the agent is homogenously distributed in the gel.

Additionally, or alternatively, one or more active agents may be combined into and/or with one or more biodegradable polymeric carrier. The term “polymeric carrier” as used herein refers to a molecule comprised of several repeating and linked chemical units, and serves as sites where one or more active agents are linked, entrapped or embedded thereto. Specifically, when the agent is at least slightly soluble (e.g., ibuprofen having a reported solubility of 21 mg/L) it may be microencapsulated in an insoluble polymeric (e.g., PLGA) particles, allowing its release in a more controlled manner.

In some embodiments, the polymeric carrier is selected from synthetic aliphatic polymer, polymers and/or copolymers.

As used hereinthroughout, the term “polymer” describes an organic substance composed of a plurality of repeating structural units (backbone units) covalently connected to one another. The term “co-polymer” as used herein, refers to a polymer of at least two chemically distinct monomeric units.

The term “embedded” refers to terms as follows: “entrapped”, “incorporated with”, “encapsulated in”, “comprises” and otherwise “provided in remote or physical connection with”, or may be considered part of the polymeric structure or the polymeric particles made therefrom.

As used herein, the term “microparticles” refers typically to particles of about 0.1 μm to about 100 μm, about 0.5 μm to about 50 μm, or about 0.5 μm to about 20 μm in at size of at least one dimension thereof.

In some embodiments, the polymeric carrier (or microparticles) comprises a polymeric backbone comprising polyester. Herein, the term “polyester” is also meant to include one monomeric unit, or oligomer comprising more than one monomeric unit, from which the corresponding polymeric backbone may, for example, be derived from ethylene glycol (derived from petroleum) and terephthalic acid. That is, in some embodiments, the polymeric backbone of the polyester comprises 1, 2, 3, 4, 5, 6, 7, 8, 9, 10, or more, monomeric units that correspond to, or are derived from, polyester. In some embodiments, at least one monomeric unit corresponds to, or derived from, polyester is not linked to another monomeric unit corresponds to, or derived from, polyester.

The term “aliphatic” with respect to a polyester means that all carbon-carbon bonds in the polyester are single bonds.

Non-limiting examples of polyester which may be used include: aliphatic polyesters, copoly(ether-esters), polyorthoesters, polyoxaesters, polyamidoesters, polyoxaesters containing amine groups, a, co-hydroxyl, carboxyl compounds, poly(lactic-co-glycolic acid) (also referred to as “PLGA”) and combinations thereof.

In sub-aspects of this embodiment, one or more therapeutic agents are loaded into the polymer matrix in respective weight ratio of, e.g., 1:1, 1:2, 1:3, 1:4, 1:5, 1:6, 1:7, 1:8, 1:9, or 1:10, including any value and range therebetween. In exemplary embodiments, the weight ratio of ibuprofen to PLGA is about 1:5, respectively.

In some embodiments, the polymeric carrier is selected from synthetic aliphatic polymers comprising one or more monomeric units selected from, without being limited thereto, D-lactic acid, L-lactic acid, lactide (including L-, D-, meso forms), glycolic acid, glycolide, caprolactone, p-dioxanone and trimethylene carbonate and mixtures or blends thereof. Biodegradable polyurethanes prepared using, but not limited to, diisocyanates such as Ethyl 2,6-diisocyanatohexanoate (ELDI) and Methyl 2,6-diisocyanatohexanoate (MLDI) together with degradable aliphatic polyester diols and with degradable chain extenders such as 2-Hydroxyethyl-2-hydroxypropanoate, 4-((1-(1-Amino-2-phenylethoxy) ethoxy)methylcyclohexyl)methyl-2-amino-3-phenylpropanoate, 1,1-(Hexane-1,6-diyl) bis(3-(2-hydroxyethylurea, Ethane-1,2-diyl bis(3-(4-hydroxyphenyl) propanoate, Bis(2-hydroxyethyl) phosphate and Bis(2-hydroxyhexyl) phosphate may also be suitable for preparing the gel carriers.

In some embodiments, the polymeric carrier comprises gelatin in the form of microparticles. As used herein the term “gelatin” also includes gelatin derivatives such as gelatin derivatized with aromatic sulfonyl chlorides, carboxylic acid chlorides, carboxylic acid anhydrides, aryl isocyanates, etc. The term “gelatin” also includes collagen and derivatives thereof.

In some embodiments, the microparticles are dispersed in a medium, e.g., water. In some embodiments, the medium further comprises an emulsifier. In some embodiments, the emulsifier is an amphiphilic emulsifier. Exemplary emulsifiers may include, without being limited thereto, polyvinyl alcohol (PVA) (e.g., MW of 6,000-20,000) and derivatives thereof.

As described herein, the active agents may be present in or combined with the polymeric carrier, e.g., microparticles, in the sealant composition or in the gel. Advantageously, the particles are homogenously distributed in the sealant composition. Typically, most of the partially soluble active agent is present in the gel embedded in polymeric carrier particles.

In one embodiment, at least part of the one or more active agents is released from the particles and/or from the gel through diffusion and/or upon biodegradation of the particles and/or of the gel.

The degradation rate of the gel may be selected to synchronize with the degradation rate of the polymeric carrier so that the drug payload is efficiently delivered in the prescribed time period for therapeutic effect. Alternatively, the degradation rate of the gel may be slower than the polymeric carrier if the gel is also required to serve as a mechanical buttress to the anastomosis beyond the treatment period required for the therapeutic effect.

In some embodiments, the flowable biocompatible sealant composition is provided in or by using a delivery system or device. Delivery system may be in the form of an applicator, e.g., a two-syringe device as described hereinthroughout.

In some embodiments, the flowable biocompatible sealant composition is provided in a delivery system in the form of component A comprising: one member selected from: electrophilic functionalized PEG, the agents and fibrinogen and component B comprising one member selected from: nucleophilic functionalized PEG and thrombin.

Embodiments of nucleophilic functionalized PEG and electrophilic functionalized PEG are described hereinthroughout.

The sealant of the invention may be applied by any means used by surgeons for treatment or prevention of defects including, but not limited to, open surgery, and minimal invasive procedure/surgery (MIS), such as in a laparoscopic procedure. In one embodiment of the invention, an incision is made at the site of surgery and the sealant is applied onto the area of the gastrointestinal surgical site. In another embodiment, an incision is made at the site of surgery, the incision is stapled or sutured, and the sealant is applied onto the area of the staple or suture line. The patient can receive local, regional or general anesthesia.

The term “open surgery” refers to surgery wherein the surgeon gains direct access to the surgical site by a relatively large incision.

As used herein the term “minimally invasive procedure” refers to a surgery wherein the surgeon gains access to the surgical site via small incisions or through a body cavity or anatomical opening e.g., via laparoscopy. Specialized techniques can be used to visualize the operated area such as, miniature cameras with microscopes, tiny fiber-optic flashlights and high-definition monitors. Instruments having “end effectors” such as forceps, cutters, needle holders, cauterizers, and the like, can be introduced to the surgical site.

Advantageously, the gel formed according to the invention releases the active agent locally for a therapeutically effective period of time. The term “therapeutically effective period of time” with respect to the administration of the composition in the method of this disclosure means the period of time sufficient to prevent or cure impaired or deteriorating function in the human or bodily site. “A therapeutically effective period of time” may be e.g., over a period of 7 to 90 days after the application e.g., 15 to 30 days.

In another aspect, the present invention provides a composition being a sealant comprising one or more active ingredients suspended in the sealant.

The biocompatible polymer matrices allow for slow, sustained or extended release of a therapeutic agent. Each therapeutic agent disclosed herein may have its own release rate at the target (e.g., GI) site.

In some embodiments, at least one portion of the composition exhibits a release profile of the agent for preventing ischemia that approximates first-order release kinetics under in vitro conditions. In some embodiments, disclosed composition releases one or more therapeutics disclosed herein (e.g., an agent for preventing ischemia) with approximate first-order release kinetics over a period of, e.g., about 7 days, about 15 days after administration, about 30 days, about 45 days, about 60 days, about 75 days, or about 90 days after administration, including any value and range therebetween. The present disclosure provides a composition being a sealant comprising one or more active ingredients combined with a polymeric carrier suspended or embedded in the sealant. As shown in the Examples section, the release profile of PLGA-Ibuprofen was consistently at an approximate first-order release profile both from a fibrin clot (see FIGS. 4A and 4B) and from a polyethylene glycol (PEG) gel (see FIGS. 6A and 6B).

In some embodiments, at least one portion of the composition exhibits a release profile of the agent for preventing infection that approximates zero-order release kinetics under in vitro conditions.

In some embodiments, the disclosed composition releases one or more therapeutics disclosed herein (e.g., agent for preventing infection) with approximate zero-order release kinetics over a period of, e.g., about 7 days, about 15 days after administration, about 30 days, about 45 days, about 60 days, about 75 days, or about 90 days after administration, including any value and range therebetween. As shown in the Examples section which follows, ciprofloxacin was consistently at an approximate zero-order release profile both from a fibrin clot (see FIGS. 3A and 3B) and from a PEG gel (see FIGS. 5A and 5B).

Zero-order release kinetics refers to the process of constant drug release from a drug delivery matrix resulting in drug blood levels that would remain constant throughout the delivery period.

Each embodiment described hereinthroughout may be incorporated in any one of the aspects relating to the compositions or the methods.

As used herein the term “about” refers to +10%.

The terms “comprises”, “comprising”, “includes”, “including”, “having”, and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, and unless stated otherwise, the terms “by weight”, “w/w”, “weight percent”, or “wt. %”, which are used herein interchangeably describe the concentration of a particular substance out of the total weight of the corresponding mixture, solution, formulation or composition.

As used herein, and unless stated otherwise, the terms “by volume”, “v/v”, “volume percent”, or “v %”, which are used herein interchangeably describe the concentration of a particular substance out of the total volume of the corresponding mixture, solution, formulation or composition.

In those instances where a convention analogous to “at least one of A, B, and C, etc.” is used, in general such a construction is intended in the sense one having skill in the art would understand the convention (e.g., “a composition comprising at least one of A, B, and C” would include but not be limited to compositions that comprise that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1: Preparation of Active Agents Embedded in Fibrin Sealant

EVICEL® Fibrin Sealant product was used in the following examples.

Poly(lactic-co-glycolic acid) (PLGA) from the Resomer RG family manufactured by Evonik was selected with lactide: glycolide ratios ranging from 50:50 to 85:15, respectively, having Inherent Viscosities (IV) ranging from 0.09 to 1.7 dL/g.

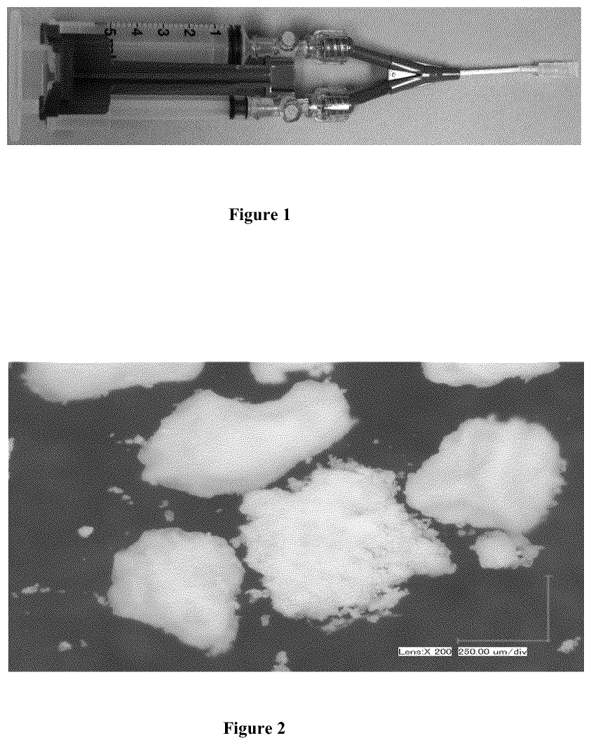

The fibrinogen and thrombin components are prepared according to the instructions provided with the product. The applicator used here is a modification of the product. The applicator that is supplied with the product is constructed with two syringes of equal volume (2 ml or 5 ml). For the purposes of this evaluation the device was modified to have a 5 ml syringe and a 1 ml syringe. The device is shown in the photographic image presented in FIG. 1. Fibrinogen was loaded into the 5 ml syringe and thrombin is loaded into the 1 ml syringe. The plunger was then pushed to apply the sealant to the area of surgical site or into a test reservoir.

Preparation of ciprofloxacin embedded in fibrin sealant: In exemplary procedures, 100 mg of ciprofloxacin were transferred into a 5 ml thawed fibrinogen component (BAC2) vial of the fibrin sealant. The fibrinogen and ciprofloxacin powder were mixed thoroughly using a vortex mixer. The drug mixed fibrinogen component was loaded into the 5 ml syringe of the sealant applicator. The thrombin solution was loaded into 1 ml syringe of the sealant applicator. The loaded syringes were mounted into the sealant applicator and the biologic components of fibrinogen containing ciprofloxacin and thrombin were applied onto the target area of the surgical site or into a test reservoir by pressing the syringe-plunger connector. Ciprofloxacin was in the form of particles in suspension in the formed gel.

Preparation of ibuprofen embedded in fibrin sealant: In exemplary procedures, 100 mg of ibuprofen powder was transferred into a 5 ml fibrinogen vial of fibrin sealant. The fibrinogen and ibuprofen powder were mixed thoroughly using a vortex mixer. The drug mixed fibrinogen component was loaded into the 5 ml syringe of the sealant applicator. Thrombin solution was loaded into 1 ml syringe of the sealant applicator. The loaded syringes were mounted into the sealant applicator and the biologic components comprising fibrinogen containing ciprofloxacin and thrombin were applied onto the target area of the surgical site or into a test reservoir by pressing the syringe-plunger connector.

Preparation of PLGA-ibuprofen particles: In exemplary procedures, 200 mg of PLGA were dissolved into 10 ml of ethyl acetate in a conical tube (15 ml) overnight. An amount 20 or 40 mg of ibuprofen was then added and let dissolve in 30-60 minutes. Next, 25 or 50 ml of polyvinyl alcohol (PVA, 1% in H₂O) emulsifier was prepared with a stirring bar at speed of 300 rpm. The mixture of PLGA-ibuprofen was then dropped into the PVA (1%) with the stirring speed of 300 rpm. Next, the polyvinyl solution was further stirred for 4 hours. Next, 1.8 ml of the particle suspension was loaded into Eppendorf tubes and followed by spinning at 10,000 rpm for 10 minutes, and thereafter the liquid was removed. Next, 400 μl of saline were added to the pellet and vortexed to suspend the particles, and the suspension was combined into 4 Eppendorf tubes, followed by spinning in centrifugation at 10,000 rpm for 10 minutes to collect all particles. Image of PLGA-ibuprofen particles under a light microscope is shown in FIG. 2, showing homogeneous PLGA-Ibuprofen Particles of around 200 to 500 μm size.

Preparation of PLGA-ibuprofen embedded in fibrin sealant: In exemplary procedures, 100 mg of PLGA-ibuprofen particles were transferred into a 5 ml fibrinogen vial of fibrin sealant. The fibrinogen and PLGA-ibuprofen powder were mixed thoroughly using a vortex mixer. The drug mixed fibrinogen component was loaded into the 5 ml syringe of the sealant applicator. Thrombin solution was loaded into 1 ml syringe of the sealant applicator. The loaded syringes were mounted into the sealant applicator and the biologic components comprising fibrinogen and thrombin were applied onto the target area of the surgical site or into a test reservoir by pressing the syringe-plunger connector.

The composition is characterized by adhesiveness within the range of 0.1 kPa to 100 kPa as measured by a tack test based on ASTM F2258-05.

### Example 2: Preparation of Active Agents Embedded in Polyethylene Glycol (PEG) Gels

Materials and Methods: PEG-SG: 4-Arm Polyethylene Glycol Succinimidyl Glutarate MW 20 kDa manufactured by JenKem Technologies

PEG-amine: 4-Arm Polyethylene Glycol Amine Hydrochloride 20 kDa manufactured by JenKem Technologies

A 6 mL PEG gel kit nominally comprised of 3.00 mL of 200 mM N-cyclohexyl-2-aminoethanesulfonic acid (CHES) buffer, 3.00 mL 9.04 mM citrate buffer, 408 mg PEG-SG powder, and 408 mg PEG-Amine powder. The buffers are packaged in glass syringes and the powders are packaged in glass vials. To prepare the device, the PEG-SG powder is dissolved in the citrate buffer and the PEG-Amine powder is dissolved in the CHES buffer, simultaneously.

Preparation of ciprofloxacin embedded in PEG gel: in exemplary procedures, 100 mg of ciprofloxacin were transferred into a 5 ml polyethylene glycol Succinimidyl Glutarate (PEG-SG) vial of synthetic sealant. The ciprofloxacin powder was mixed thoroughly using a vortex mixer. The drug mixed PEG-SG component was loaded into the 5 ml syringe of the sealant applicator, and PEG-amine solution was loaded into another 5 ml syringe of the sealant applicator. The PEG components of PEG-SG and PEG-amine were applied onto the target area of the surgical site or into a test reservoir by pressing the syringe-plunger connector.

Preparation of ibuprofen embedded in PEG gel: In exemplary procedures, 100 mg of ibuprofen were weighed and transferred into a 5 ml PEG-SG synthetic sealant vial. Next, 5 ml of PEG-SG and ibuprofen powder were mixed using a vortex mixer. The drug mixed PEG-SG component was then into the 5 ml syringe of the sealant applicator. The PEG-amine solution was loaded into another 5 ml syringe of the sealant applicator. Next, the syringes were mounted into the sealant applicator, and the PEG components of PEG-SG and PEG-amine were applied onto the target area of the surgical site or into a test reservoir by pressing the syringe-plunger connector.

Preparation of PLGA-ibuprofen particles: see Example 1.

Preparation of PLGA-ibuprofen embedded in PEG gel; In exemplary procedures, 100 mg of PLGA-ibuprofen were weighed, and transferred into a 5 ml PEG-SG vial of synthetic sealant.

Next, 5 ml of PEG-SG and PLGA-ibuprofen powder were mixed using a vortex mixer. The mixed PEG-SG component was loaded into the 5 ml syringe of the sealant applicator, and then PEG-amine solution was loaded into another 5 ml syringe of the sealant applicator. The syringes were then mounted into the sealant applicator, and the PEG components of PEG-SG and PEG-amine were then applied onto the target area of the surgical site or into a test reservoir by pressing the syringe-plunger connector.

The composition is characterized by adhesiveness within the range of 0.1 kPa to 100 kPa as measured by a tack test based on ASTM F2258-05.

### Example 3: Release of Active Agents from Fibrin Clot in an In Vitro Setting

Release of ciprofloxacin from fibrin clot in an in vitro setting: Ciprofloxacin embedded in fibrin sealant was prepared as described above and the mixture was injected into a 1 ml tube or cup and was let to clot for 2 hours at room temperature. The formed clot (1 ml gel) was transferred into a 15 ml conical tube and 10 ml of saline were then added. The tube was put on a shaker and the 10 ml solution was collected followed by replacing with another 10 ml saline next day. This step was repeated 14 times (days).

Ciprofloxacin concentration was measured by fluorescence of excitation at 277 nm and emission at 418 nm.

Individual daily amount and total amount of ciprofloxacin (“cipro”) released in the solution is listed in Table 1 below (see FIGS. 3A-B).

TABLE 1

| Day | μg | Total, μg |
|---|---|---|
| 1 | 617.7 | 616.7 |
| 2 | 651.8 | 1268.5 |
| 3 | 691.6 | 1960.0 |
| 4 | 740.6 | 2700.7 |
| 5 | 836.6 | 3537.3 |
| 6 | 858.4 | 4395.7 |
| 7 | 813.2 | 5208.9 |
| 8 | 688.1 | 5897 |
| 9 | 714.1 | 6611.1 |
| 10 | 744.1 | 7355.2 |
| 11 | 559.1 | 7914.3 |
| 12 | 673.7 | 8588 |
| 13 | 744.7 | 9332.7 |
| 14 | 820.6 | 10153.3 |

The results show that cipro was consistently released into the solution at the concentration of about 50 to about 80 μg/ml/day for 14 days, demonstrating a zero-order release profile.

Release of ibuprofen from fibrin clot in an in vitro setting: A similar procedure was applied for ibuprofen embedded in fibrin sealant and for PLGA-ibuprofen embedded in fibrin sealant. The formed clot (1 ml) was transferred into a 15 ml conical tube and 10 ml of saline were then added. The tube was put on a shaker and the 10 ml solution was collected followed by replacing with another 10 ml saline next day. This step was repeated 15 times (days). The ibuprofen concentration was measured by its UV signature, that is, UV absorption at 221 nm. Individual daily amount and accumulative amount of ibuprofen released in the solution is listed in Table 2A (ibuprofen embedded in fibrin sealant) and Table 2B (for PLGA-ibuprofen embedded in fibrin sealant; see also FIGS. 4A-B).

TABLE 2A

| Day | μg | Total, μg |
|---|---|---|
| 1 | 1849 | 1849 |
| 2 | 840 | 2689 |
| 3 | 751 | 3441 |
| 4 | 729 | 4170 |
| 5 | 700 | 4870 |
| 6 | 481 | 5351 |
| 7 | 366 | 5716 |
| 8 | 244 | 5960 |
| 9 | 278 | 6238 |
| 10 | 343 | 6581 |
| 11 | 135 | 6716 |
| 12 | 101 | 6817 |
| 13 | 81 | 6898 |
| 14 | 54 | 6952 |

TABLE 2B

| Day | μg | Total, μg |
|---|---|---|
| 0 | 230.7 | 230.7 |
| 1 | 2650.8 | 2881.4 |
| 2 | 769.3 | 3650.8 |
| 3 | 204.9 | 3855.7 |
| 4 | 100.5 | 3956.2 |
| 5 | 69.6 | 4025.8 |
| 6 | 61.9 | 4087.6 |
| 7 | 59.3 | 4146.9 |
| 8 | 45.1 | 4192 |
| 9 | 78.6 | 4270.6 |
| 10 | 46.4 | 4317 |
| 12 | 51.5 | 4368.6 |
| 13 | 52.8 | 4421.4 |
| 14 | 51.5 | 4472.9 |
| 15 | 50.3 | 4523.2 |

The results show the ibuprofen releasing in a less than controlled manner when placed neat (without PLGA microencapsulation).

### Example 4: Release of Active Agents from Peg Gels in an In Vitro Setting

Release of ciprofloxacin from PEG gel in an in vitro setting: Ciprofloxacin embedded in PEG gel was prepared as described above. In exemplary procedures, the syringes were mounted into the sealant applicator and the PEG mixture was injected into a 1 ml tube or cup and was kept for 2 hours at room temperature to allow the crosslinking. Next, the formed gel (1 ml) was transferred into a 15 ml conical tube and 10 ml of saline were then added. The tube was put on a shaker and the 10 ml solution was collected followed by replacing with another 10 ml saline next day. This step was repeated 14 times (days). The ciprofloxacin concentration was measured by fluorescence of excitation at 277 nm and emission at 418 nm. Individual daily amount and accumulative amount of ibuprofen released in the solution is listed in Table 3 (see FIGS. 5A-B).

TABLE 3

| Day | μg | Total, μg |
|---|---|---|
| 1 | 425.0 | 425.0 |
| 2 | 369.3 | 794.3 |
| 3 | 256.8 | 1051.1 |
| 4 | 202.8 | 1254.0 |
| 5 | 157.6 | 1411.6 |
| 6 | 154.6 | 1566.2 |
| 7 | 85.9 | 1652.1 |
| 8 | 143.7 | 1795.8 |
| 9 | 115.3 | 191.11 |
| 10 | 119.7 | 2030.9 |
| 11 | 113.6 | 2144.4 |
| 12 | 113.0 | 2257.4 |
| 13 | 114.8 | 2372.2 |
| 14 | 111.9 | 2484.1 |

The results show that cipro was consistently released into the solution at the concentration of about 10 to about 40 μg/ml/day for 14 days, exhibiting a zero-order release profile.

Release of ibuprofen from PEG gel in an in vitro setting: A similar procedure was applied for ibuprofen embedded in PEG gel and for PLGA-ibuprofen embedded in PEG gel. The formed clot (1 ml) was transferred into a 15 ml conical tube and 10 ml of saline were then added. The tube was put on a shaker and the 10 ml solution was collected followed by replacing with another 10 ml saline next day. This step was repeated 14 times (days). The ibuprofen concentration was measured by UV absorption at 221 nm. Individual daily amount and accumulative amount of ibuprofen released in the solution is listed in Table 4A (ibuprofen embedded in PEG gel) and Table 4B (for PLGA-ibuprofen embedded in PEG gel; see also FIGS. 6A-B).

As for the release profile of PLGA-Ibuprofen from PEG gels, based on the Ibuprofen UV signature as measured in the release profile it can be seen that Ibuprofen was released into the solution with the highest concentration at day 1. Then the release of Ibuprofen decreased significantly day by day in the period of 15 days, exhibiting a first-order release profile.

TABLE 4A

| Day | μg | Total, μg |
|---|---|---|
| 1 | 4304 | 4304 |
| 2 | 693 | 4997 |
| 3 | 362 | 5360 |
| 4 | 157 | 5517 |
| 5 | 129 | 5646 |
| 6 | 77 | 5723 |
| 7 | 68 | 5791 |
| 8 | 135 | 5927 |
| 9 | 52 | 5978 |
| 10 | 43 | 6021 |
| 11 | 67 | 6088 |
| 12 | 59 | 6147 |
| 13 | 41 | 6188 |
| 14 | 41 | 6229 |

TABLE 4B

| Day | μg | Total, μg |
|---|---|---|
| 0 | 344 | 344 |
| 1 | 2887 | 3231 |
| 2 | 954 | 4184 |
| 3 | 331 | 4515 |
| 4 | 168 | 4683 |
| 5 | 95 | 4778 |
| 6 | 102 | 4880 |
| 7 | 53 | 4933 |
| 8 | 1 | 4934 |
| 9 | 18 | 4952 |
| 10 | 67 | 5019 |
| 12 | 6 | 5026 |
| 13 | 0 | 5026 |
| 14 | 1 | 5027 |
| 15 | 1 | 5028 |

### Example 5: Ciprofloxacin Concentration Testing

The purpose of this study was to determine if Ciprofloxacin released from PEG gel and Fibrin clot was active against challenge bacteria:
Escherichia coli ATCC 25922
Staphylococcus aureus ATCC 6538

Inoculum preparation: E. coli-one colony was taken from 1^{st} pass Tryptic Soy Agar (TSA) streak plate onto TSA plate (2^{nd} pass). Plates were incubated at 35±2° C. for 18-24 hours. One isolated colony from the 2^{nd} pass plate was taken with sterile loop and transferred into 5 ml of Mueller Hinton Broth (MHB) tube. The broth was incubated at 37±2° C. for 2-6 hours.

The tubes were then removed from the incubator. The inoculum was adjusted to 0.5 McFarland by diluting the 2-6-hour culture in sterile (deionized) DI water using Biomerieux DensiCHEK Plus (0.44-0.56) to achieve a final concentration of 1-2×10⁸ CFU/ml. 0.5 ml of the adjusted inoculum was transferred to 49.5 ml of MHB broth.

S. aureus-one colony was taken from 1^{st} pass streak plate onto TSA plate (2^{nd} pass). Plates were incubated at 3δ+2° C. for 18-24 hours.

The streak plates were removed from incubator, and 2-3 colonies from the streak plate were transferred to 2 ml of sterile DI water using sterile loop. The inoculum was adjusted to 0.5 McFarland by diluting the 2-6-hour culture in sterile DI water using Biomerieux DensiCHEK Plus (0.44-0.56) to achieve a final concentration of 1-2×10⁸ CFU/ml. Next, 0.5 ml of the adjusted inoculum was transferred to 49.5 ml of MHB broth.

Sample Preparation: Samples and standard of 16 μg/ml were prepared, see Table 5. All samples were stored in refrigerator between 4°-8° C.

TABLE 5

| | PEG gel | | Fibrin Clot |
|---|---|---|---|
| No | (μg/ml) | No | (μg/ml) |
| P0 | 14.9 | F0 | 7.4 |
| P1 | 29.2 | F1 | 33.7 |
| P2 | 28.6 | F2 | 30.8 |
| P3 | 15.3 | F3 | 22.2 |
| P4 | 18.0 | F4 | 24.9 |
| P5 | 13.7 | F5 | 26.7 |
| P6 | 13.3 | F6 | 25.3 |
| P7 | 14.7 | F7 | 26.5 |
| P8 | 11.2 | F8 | 27.1 |
| P9 | 16.8 | F9 | 24.7 |
| P10 | 15.2 | F10 | 23.4 |
| P11 | 9.5 | F11 | 25.3 |
| P12 | 11.4 | F12 | 23.3 |
| P13 | 10.0 | F13 | 29.8 |
| P14 | 16.3 | F14 | 27.8 |
| P15 | 12.9 | F15 | 30.8 |

Test Details: Test plates—The test was performed in a 96 well microtiter plate. A total of 4 well plates were used for test per organism per source of release (PEG gel or Fibrin clot). Sterile MHB was poured into a reservoir and dispensed in 50 μl aliquots into wells 1-12 for all 16 well plates. An amount of 50 μl of concentrations of ciprofloxacin (P0-P15 or F0-F15) were dispensed into the first column of microtiter plate in duplicate (For example: A1, B1 contained P0 sample). Next, 1:2 serial dilution was performed in the well plate by taking 50 μl from first column into the second column up to column 11 using multichannel pipettor. This step was performed for all 16 well plates. 50 μl of the adjusted inoculum prepared in MHB for E. coli or S. aureus was dispensed in all wells of the 16 microtiter plates.

Standard Plate: One microtiter plate was used to test the standard provided of 16 μg/ml. The standard was tested in duplicate per organism. 50 μl of MHB was dispensed from wells 1-12. An amount of 50 μl of the standard was transferred into column 1, rows A-D. Next, 1:2 serial dilution was performed in the well plate by taking 50 μl from first column to second column up to column 11 using multichannel pipettor. An amount of 50 μl of the adjusted inoculum prepared in MHB for E. coli or S. aureus were dispensed in all columns.

Negative Control: To rule out any cross contamination, negative control plate was performed. Two replicates per organism were performed. Next, 50 μl of MHB was dispensed in columns 1-11 and 100 μl of MHB was dispensed in column 12. Next, 50 μl of the adjusted inoculum prepared in MHB for E. coli or S. aureus were dispensed in columns 1-11, rows A-D. Column 12 did not get any inoculum, to confirm the sterility of the test.

Inoculum counts: To confirm the inoculum count, 0.01 ml aliquot was removed from growth control well (Column_12) from any test plate and was transferred to 10 ml of sterile MHB. 0.1 ml from the tube was plated in triplicate for each organism and poured with TSA. All well plates were sealed using a sterile breathable rayon film to prevent drying. Well plates and inoculum count plates were incubated at 37±2° C. for 16-20 hours.

Results: Table 6 presents the Inoculum Counts. Table 7 presents the active ciprofloxacin concentration in PEG gel and Fibrin Clot, and Table 8 presents the Standard Concentration and minimum inhibitory concentration (MIC) of Ciprofloxacin (μg/ml) for each bacterium.

TABLE 6

| Organism | Average CFU/mL |
|---|---|
| E. coli 25922 | 3.8 × 10⁵ |
| S. aureus 6538 | 4.8 × 10⁵ |

TABLE 7

| | | Active | | | | Active | |
|---|---|---|---|---|---|---|---|
| | | Ciprofloxacin | | | | Ciprofloxacin | |
| | PEG | (μg/ml) | | | Fibrin | (μg/ml) | |
| | gel | E. | S. | | Clot | E. | S. |
| No | (μg/ml) | coli | aureus | No | (μg/ml) | coli | aureus |
| P0 | 14.9 | 0.014 | 0.2 | F0 | 7.4 | 0.029 | 0.2 |
| P1 | 29.2 | 0.014 | 0.1 | F1 | 33.7 | 0.016 | 0.1 |
| P2 | 28.6 | 0.014 | 0.2 | F2 | 30.8 | 0.015 | 0.1 |
| P3 | 15.3 | 0.015 | 0.2 | F3 | 22.2 | 0.011 | 0.0 |
| P4 | 18.0 | 0.035 | 0.1 | F4 | 24.9 | 0.012 | 0.0 |
| P5 | 13.7 | 0.027 | 0.2 | F5 | 26.7 | 0.013 | 0.1 |
| P6 | 13.3 | 0.026 | 0.2 | F6 | 25.3 | 0.012 | 0.0 |
| P7 | 14.7 | 0.029 | 0.4 | F7 | 26.5 | 0.013 | 0.1 |
| P8 | 11.2 | 0.044 | 0.1 | F8 | 27.1 | 0.013 | 0.1 |
| P9 | 16.8 | 0.033 | 0.1 | F9 | 24.7 | 0.012 | 0.0 |
| P10 | 15.2 | 0.030 | 0.2 | F10 | 23.4 | 0.011 | 0.0 |
| P11 | 9.5 | 0.018 | 0.2 | F11 | 25.3 | 0.012 | 0.0 |
| P12 | 11.4 | 0.022 | 0.3 | F12 | 23.3 | 0.011 | 0.1 |
| P13 | 10.0 | 0.019 | 0.1 | F13 | 29.8 | 0.014 | 0.1 |
| P14 | 16.3 | 0.016 | 0.2 | F14 | 27.8 | 0.013 | 0.1 |
| P15 | 12.9 | 0.025 | 0.4 | F15 | 30.8 | 0.015 | 0.1 |

TABLE 8

| Standard | MIC for Ciprofloxacin | |
|---|---|---|
| Ciprofloxacin | (μg/ml) | |
| (μg/ml) | E. coli | S. aureus |
| 16.0 | 0.016 | 0.125 |

The sterility control plate is presented in Table 9.

TABLE 9

| | |
|---|---|
| E. coli ATCC 25922 | Pass |
| S. aureus ATCC 6538 | Pass |

Based on the results, it can be concluded that the ciprofloxacin is still active in the samples released from PEG gel and Fibrin Clot and is effective against the E. coli and S. aureus strains.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A flowable biocompatible sealant composition in the form of a biodegradable cross-linked gel, the composition comprising an insoluble agent for preventing infection, and an agent for preventing ischemia; wherein:
(a) the agent for preventing ischemia is embedded in polymeric microparticles;
(b) the composition is characterized by adhesiveness within the range of 0.1 kPa to 100 kPa as measured by a tack test based on ASTM F2258-05, and wherein:
(c) the composition is characterized in that it releases at least part of said agents in a sustained manner.

2. The composition of claim 1, wherein the gel comprises fibrin.

3. The composition of claim 1, wherein the gel comprises functionalized polyethylene glycol (PEG).

4. The composition of claim 1, wherein the functionalized PEG is in a multi-arm form.

5. The composition of claim 1, wherein the insoluble agent for preventing infection comprises ciprofloxacin.

6. The composition of claim 1, wherein the agent for preventing ischemia comprises nonsteroidal anti-inflammatory agent.

7. The composition of claim 6, wherein the anti-inflammatory agent comprises ibuprofen.

8. The composition of claim 1, wherein the agent for preventing ischemia is homogenously embedded in the polymeric microparticles.

9. The composition of claim 1, wherein the polymeric microparticles are made of PLGA or gelatin.

10. The composition of claim 1, comprising at least one portion exhibiting a release profile of the agent for preventing ischemia that is representative of first-order release kinetics under in vitro conditions.

11. The composition of claim 1, comprising at least one portion exhibiting a release profile of the agent for preventing infection that is representative of zero-order release kinetics under in vitro conditions.

12. A method for preventing and/or treating leakage from an anastomosis site in a subject in need thereof, said method comprising the steps of:
(a) providing a flowable biocompatible sealant composition capable of forming a biodegradable gel, the composition comprising a therapeutically effective amount of an agent for preventing infection, and an agent for preventing ischemia;
(b) applying said composition of step (a) to the anastomosis site; and
(c) allowing the composition to form a gel, and to locally release at least part of the two active agents through diffusion and/or upon biodegradation of said gel.

13. The method of claim 12, wherein the anastomosis site is within the gastrointestinal tract

14. The method of claim 12, wherein the agent for preventing ischemia is embedded in polymeric microparticles

15. The method of claim 12, wherein the sealant composition is selected from fibrin and Polyethylene glycol (PEG).

16. The method of claim 12, wherein the PEG is in a multi-arm form.

17. The method of claim 12, being in a local administration.

18. The method of claim 12, wherein the flowable biocompatible sealant composition is provided in or by using a delivery system.

19. The method of claim 12, wherein the flowable biocompatible sealant composition is provided in a delivery system in the form of component A comprising: one member selected from: electrophilic functionalized PEG, said agents and fibrinogen and Component B comprising one member selected from: nucleophilic functionalized PEG and thrombin.

20. The method of claim 12, wherein the electrophilic functionalized PEG has one or more succinimidyl glutarate groups and the nucleophilic functionalized PEG has one or more amine groups.
